(19) United States
(12) Patent Application Publication
(10) Pub. No.: US 2020/0108105 A1
(43) Pub. Date: Apr. 9, 2020

(54) MICROCAPSULES CONTAINING LIVE MICROORGANISMS AND USE THEREOF

(71) Applicant: CosmoLab LLC., Novosibirsk (RU)

(72) Inventor: Artem Mihajlovich GURIEV, Tomsk (RU)

(21) Appl. No.: 16/314,144

(22) PCT Filed: May 22, 2017
(86) PCT No.: PCT/RU2017/000328

§ 371 Date: Dec. 28, 2018

(30) Foreign Application Priority Data
Apr. 13, 2017 (RU) 2017112773

(51) Int. Cl. A61K 35/741; A61K 9/50; A61K 8/99; A61K 8/11; A61K 36/064; A61K 35/747; A61K 35/745

(52) U.S. Cl. CPC A61K 35/741; A61K 9/50; A61K 8/99; A61K 35/745; A61K 36/064; A61K 9/5089; A61K 35/747; A61K 8/11

(57) ABSTRACT

A microcapsule for a topical treatment for improving skin and mucus membranes condition includes a matrix material in a solid state, in which a live probiotic microorganism is encapsulated, wherein the live microorganism is released from the matrix upon contact with a skin surface and/or mucous membranes of the human or warm-blooded animal body, wherein the matrix material has a melting or softening temperature selected from the range of 20-43° C., and wherein the live probiotic microorganism is present in the range of 0.001 to 80 wt. % from the weight of the matrix material.

This application is based on a 371 of PCT/RU2017/000328, filed May 22, 2017, which claims benefit of Russian Application No. 2017112773 filed on Apr. 13, 2017.

### FIELD OF THE INVENTION

The invention relates to therapeutic and cosmetic products for external use and can be used in medicine and cosmetology to normalize the microflora, homeostasis and barrier function of the skin and mucous membranes during aging, damage by environmental factors (UV rays, wind, low temperatures, injuries) and pathological processes (microbial infections, inflammatory and allergic diseases, metabolic disorders).

### BACKGROUND OF THE INVENTION

Normal microflora plays an important role in the formation of a protective barrier in case of contamination of the skin and mucous membranes by pathogenic microorganisms (bacteria, fungi, viruses) and in recovery processes for injuries of various etiology. In addition, the composition of microflora affects the intensity of the processes of natural aging of the skin, maintaining its homeostasis, fluid and electrolyte balance, turgor and other indicators of the performance status.

The positive effect of probiotic microorganisms is evidenced by the numerous literature data and the presence of a sustainable market for a variety of products containing live bacteria. Probiotics for the treatment and prevention of gastrointestinal diseases, intestinal dysbioses, and vaginoses are common and constitute a large segment of the market. However, the direction of the use of microorganisms in external dosage forms and in cosmetics is only beginning to develop and is extremely promising.

Currently, there is a need for new cosmetic products that provide the possibility of applying microorganisms to the skin and/or mucous membranes.

There are a large number of cosmetics, the effect of which is based on the use of metabolic by-products (metabolism), as well as the products of lysing of microorganisms (lysates), disclosed, for example, in the following patent documents: WO2011026039, CN102225045, U.S. Pat. Nos. 8,715,650, 6,645,506, 7,651,680, US20050201996, U.S. Pat. No. 8,709,454.

Cosmetics containing live microorganisms or their viable spores are also known, for example, the following patent documents: US20130251695, US20090186057, U.S. Pat. No. 8,709,454, WO0113927.

However, as a rule, such products have unsatisfactory consumer properties, such as low stability and short shelf life, unpleasant odor, which are caused by the vital processes of microorganisms that interact with water and other active substances contained in the pharmaceutical and cosmetic compositions.

To isolate microorganisms from interaction with the environment, their microencapsulation can be used.

A composition in the form of microencapsulated lactobacilli is known from U.S. Pat. No. 5,614,209 A (publ. 25 Mar. 1997), which also describes a method for producing microencapsulated lactobacilli using a process for removing a highly volatile solvent from a solution of copolymers of acrylic and methacrylic acids.

Microcapsules having the form of gray-yellow particles ranging in size from 10 to 200 μm, which contain lyophilized culture of lactobacilli, are known from the patent RU 2220716 C1 (publ. 10 Jan. 2004). The method for preparation microcapsules consists of covering the lyophilized culture of lactobacilli with a shell that contains polyvinylpyrrolidone tanned with tannin.

Microencapsulated forms of microorganisms using a copolymer of acrylic and methacrylic acids in the form of an aqueous suspension are known from the patent RU 2171672 C1 (publ 10 Aug. 2001). As a technological environment for preparation such microcapsules, paraffins, mineral and vegetable oils are used, which are removed at the final stages of the process and are not included in the composition of the prepared microcapsules.

The disadvantages of the above microcapsules is that they cannot be used as an external pharmaceutical or cosmetic product due to the fact that the solid framework base formed as a result of the production of polymeric microcapsules does not allow their disintegration (dissolution, melting, softening) at a body temperature of human and releasing of active microorganisms, when applied to the skin. The presence of polymer microcapsules, which have the form of solid indestructible particles, in the composition of a cosmetic product causes an abrasive effect when applied to the skin, because the polymeric material of the shell does not dissolve, even if the capsule is mechanically destroyed. Also, a significant drawback of the above technical solutions is that the resulting microcapsules do not protect the microorganisms contained in them against interaction with the aqueous medium and other active substances contained in the pharmaceutical and cosmetic compositions.

It is known that lipid or wax microcapsules are used to protect the active components, including bacteria, from exposure to atmospheric oxygen and moisture. Publications Santo Scalia, Paul M Young & Daniela Traini “Solid lipid microparticles as an approach to drug delivery” (Expert Opin. Drug Deliv. (2015) 12 (4):583-599); Surajit Das, Anumita Chaudhury “Recent Advances in Lipid Nanoparticle Formulations with Solid Matrix for Oral Drug Delivery” (AAPS PharmSciTech, (2011) 12(1):62-76); WO2009046930 A1 (publ. 16 Apr. 2009) disclosed lipid or wax capsules, or microcapsules, or nanocapsules with or without a shell, by matrix or reservoir type and methods for preparation thereof.

Method for preparation soft gel capsules containing a suspension of probiotic bacteria microencapsulated in a plant lipid (with a melting point of 35° C. to 75° C.) is known from US Pat. No. 9,427,012 B2 (publ. 30 Aug. 2016). The microcapsules with a size of 150-550 μm having the gel shell and stable for at least 24 months at room temperature were obtained.

Suspension of microorganisms in a non-aqueous filler, which has the form of mixture of wax, fatty oil and emulsifier is also known from the publications: WO2008046625 A2 (publ. 24 Apr. 2008) and “The Theory and Practice of Industrial Pharmacy” By Lachman and Lieberman (3rd Editn, 1987, p. 405); as well as soft or hard gelatin capsules containing said suspension, which are intended for use, for example, in areas such as food processing, feed production, cosmetic or in the manufacture of food additives. Known capsules as well as those described above in U.S. Pat. No. 9,427,012 B2 have a shell to preserve the viability of microorganisms during storage. However, the conditions that determine the possibility of cosmetic use both suspension and capsules containing thereof are not disclosed in WO2008046625 A2. “The Theory and Practice of Industrial Pharmacy” on page 398 describes the possible use of a gel capsule as a package of single dose of a cosmetic product in the form of suspension.

The disadvantages of the above-described soft and hard gelatin capsules in the context of their possible external use (including cosmetic) is the presence of a strong shell that prevents their rapid disintegration when directly applied to the skin, and possible abrasive effect when rubbed.

All the above-described known microcapsules and capsules containing microorganisms are aimed at solving the problem of protecting biologically active components (including microorganisms) from environmental exposure and increasing their stability during storage.

However, presence in microcapsules of protective properties only is not always sufficient for external use: along with this, microcapsules must have the properties that allow to ensure effective delivery of viable microorganisms to the skin and mucous membranes, as well as acceptable consumer properties.

In patent RU 2306132 C2 (publ. 20 Sep. 2007) an attempt was made to solve the effective delivery of viable microorganisms to the skin. A skin care product in the form of a tissue impregnated with a suspension of bacteria in a liquid lipid is provided that allows lactic acid producing bacteria to be delivered to the skin.

This form has a narrow application and is not related to microcapsules or cosmetic compositions containing thereof, which are intended for cosmetic use.

Therefore, there is a need to develop a therapeutic or cosmetic product having a form suitable both for self-use as an external agent and for the introduction into the composition for external use and allowing to solve the problem of efficient delivery of viable microorganisms to the skin and mucous membranes of human and warm-blooded animals.

### DISCLOSURE OF THE INVENTION

The basis of the invention is the problem of developing microcapsules containing microorganisms, and capable of effectively and controlled releasing upon contact with the skin and mucous membranes of human or warm-blooded animal.

The technical result is to ensure the possibility of developing a new form of therapeutic or cosmetic product for external use containing microorganisms with improved consumer properties: efficient delivery of viable microorganisms to the skin and mucous membranes (rapid disintegration of microcapsules upon contact with the skin or mucous membranes), no unpleasant smell, no abrasive effect.

Another goal is to improve usability and increase the shelf life without losing the viability of microorganisms.

Another goal is to ensure the stability of microcapsule in the conditions of its occurrence in gels, creams, etc.

The posed problem is solved by the fact that the claimed microcapsules consist of a matrix in which microorganisms are encapsulated wherein the matrix material has the property of melting or softening at a temperature selected from the range of 25-43° C., and the number of encapsulated microorganisms is in the range of 0.001 to 80 wt. % of the total weight of the matrix, wherein the microcapsules are capable to release the microorganisms contained in them upon contact with the skin surface or mucous membranes of the human or warm-blooded animal body.

In addition, water content in the microcapsule does not exceed 10 wt. %, preferably not more than 5 wt. %, or most preferably does not exceed 1 wt. %.

The presence of water in the matrix material in the amount of more than 10 wt. % may decrease the viability of microorganisms contained in microcapsules.

In addition, the matrix material is selected from the group of substances including: lipids: animal and vegetable oils and fats, fully hydrogenated or partially hydrogenated vegetable and animal oils and fats, saturated and unsaturated fatty acids, partially hydrogenated or fully hydrogenated fatty acids, fatty acid esters, saturated and unsaturated, partially hydrogenated or fully hydrogenated monoglycerides, diglycerides and triglycerides, phospholipids, lecithins, partially hydrogenated or fully hydrogenated phospholipids and lecithins, lysolecithins and lysophosphatidylcholine; waxes: animal waxes, plant waxes, mineral waxes, synthetic waxes, wax esters, saturated and unsaturated fatty alcohols, fatty alcohol ethers/esters; saturated and unsaturated hydrocarbons (paraffins); silicones and ethers/esters of silicones; polyol ethers/esters: glycerol ethers/esters, sorbitan, sorbitan stearate, glyceryl ricinoleate; polyglycerols and their ethers/esters; hydrophobic gelling agents: silicon dioxide, polyethylenes.

The matrix material can be selected as a mixture of the above substances.

The melting or softening point in the range of 20-43° C. is selected in each case by one skilled in the art taking into account the choice of the matrix material or mixture components with relation to their ratios.

Normal temperature in different parts of human skin and mucous membranes varies in the range of 25-37.5° C. In warm-blooded animals (cats, dogs, ungulates, etc.), the body temperature is normally 37-39° C.; birds have a body temperature of 40-43° C.

For external use of microcapsules, it is desirable that the melting or softening point of the matrix material is in the range of 26.5-35.0° C., since this is the range of the surface temperature of human skin: the average temperature on skin of the forehead of human is 33.2° C.; on the chest −33.5° C.; on the hands −30.4° C.; on the feet 26.5-27.0° C. For the rapid destruction of microcapsules when applied to the skin or mucous membranes, the melting point of the matrix material may be 3-5° C. lower than the temperature of the target body region. However, at the melting point of the matrix material below 20° C., the microcapsules will have low stability and may disintegrate during storage at room temperature.

In addition, the microcapsules have a size of 100-7000 μm and are prepared by mechanical grinding of a cooled suspension of microorganisms in the matrix material.

In addition, microcapsules have a size of 50-3000 μm and are prepared by cooling of droplets of a suspension of microorganisms in the matrix material.

In addition, microcapsules have a size of 100-2000 μm and are prepared by spraying the molten matrix material into the fluidized bed of the lyophilisate of microorganisms.

In addition, the microcapsules have a size of 20-1000 μm and are prepared by cooling an emulsified suspension of microorganisms in the matrix material.

In addition, the microcapsules have a size of 20-1000 μm and are prepared by cooling the sprayed suspension of microorganisms in the matrix material.

In addition, microcapsules have a size of 250-5000 μm and are prepared by hot extrusion of a suspension of microorganisms in the matrix material.

It is possible that microcapsules contain at least one shell and/or coating that melt or disintegrate when microcapsules are applied to the skin or mucous membranes.

The presence of such a shell, in some cases, can prevent the melting, sticking or aggregation of microcapsules when exposed to temperatures above the melting point of the matrix and thus improve consumer properties of microcapsules and their storage stability, along with the preservation of their ability to disintegrate when applied to the skin. Also the need for such a shell, in some cases, may be due to the need for additional protection of the content of the microcapsules from exposure to atmospheric oxygen, water, or components of the compositions, which may contain the microcapsules of the present invention. Especially, the presence of a shell may be relevant if the composition for external use contains substances with antimicrobial activity (for example, essential oils, tannins, terpenoids, etc.) along with the microcapsules.

Microcapsules with a shell can be prepared by spraying the molten shell material on the microcapsules, for example, in a fluidized bed unit or a drum coater.

It is desirable that the melting or softening point of the shell material is in the range of 28-72° C., preferably 35.5-54° C.

Preferably, shell material is selected from the group comprising the same substances as the matrix material, wherein the melting point of the shell material may be the same as the melting point of the matrix material.

In addition, the shell material may have a melting point exceeding the melting point of the matrix.

When using substances with a melting point exceeding the melting point of the matrix material as the shell material, an increase in the strength of the microcapsules can be achieved.

Said shell may not melt under the action of body temperature, but it may disintegrate at the time of application microcapsules on the surface of the skin or mucous membranes, when the internal content of the microcapsule (matrix) melt or soften under the action of body temperature and microcapsules will be exposured to a small mechanical effect—rubbing on the skin.

Microcapsules with a shell can also be prepared by spraying a solution or suspension of the shell material on the microcapsules, for example, in a fluidized bed apparatus or drum coater.

In this case, the shell material is selected from the group comprising the same substances as the matrix material or from the group including: cellulose ethers: hydroxymethylpropylcellulose (HPMC) and its derivatives, hydroxypropylcellulose, methylcellulose, ethylcellulose, carboxymethylcellulose (CMC), cellulose acetate phthalate, methacrylic acid and its derivatives (Eudragit), polyvinylpyrrolidone and its derivatives, polysaccharides and their derivatives: sodium alginate, gum arabic, gellan gum, starch, modified starch, guar gum, pectin, amidated pectin, carrageenan, chitosan, mesquite gum, agar gum, psyllium gum, tamarind gum, xanthan, locust bean gum; protein: wheat protein, soy protein, sodium caseinate, gelatin, zein, shellac, hyaluronic acid, its derivatives, any synthetic and natural water-soluble polymers, and mixtures thereof.

To ensure the ability of microcapsules to disintegrate when applied to the skin, it is desirable that the amount of shell material does not exceed 50% of the total weight of microcapsules.

In cases where a shell material with a melting point exceeding the melting point of the matrix is used or substances that do not melt are used, it is desirable that the amount of the shell material does not exceed 20% of the total weight of microcapsules.

The coating, as well as the shell, provides increased strength of the microcapsules and additionally provides them with anti-adhesive properties.

The coating is obtained by powdering the microcapsules with a dry micronized substance (powder).

Micronized coating material is preferably selected from the group of substances comprising the same substances as the matrix material or shell material, or substances selected from the group comprising: inorganic salts, metal oxides, talc, salts and esters of saturated and unsaturated fatty acids (for example, magnesium stearate, calcium stearate, glycerol mono- and distearate).

To achieve the protective, strength and anti-adhesive properties of microcapsules, combinations of shell and coating materials can be used, which can be applied to the microcapsule in any sequence.

Some materials (substances) of the matrix or shell, as well as some types or strains of microorganisms may be sensitive to the effects of atmospheric oxygen. Therefore, the process for preparation microcapsules can be carried out under conditions that prevent the interaction of microcapsule components with atmospheric oxygen.

Additionally, a coloring matter suitable for use in pharmaceutical, food and cosmetic products can be added to the matrix material or shell material.

In addition, the composition of microcapsules may include substances commonly added to skin care products such as surface active agents, water absorbing agents, buffering agents maintaining pH level (weak organic or inorganic acids, such as lactic acid, ascorbic acid, citric acid or boric acid), aromatics, antioxidants (such as plant extracts, flavonoids, tocopherol, retinol, β-carotene, etc.).

Composition of microcapsules may also include active agents from the list: analgesic, antiparasitic, antifungal, antiviral, anesthetic, anti-psoriatic, antipruritic, keratolytic, anti-seborrheic, anti-acne, antidermatitis, depigmenting, antihistaminic, wound healing, immunomodulatory, steroidal and non-steroidal anti-inflammatory active agents, free radical traps, anti-dandruff agents, anti-irritant agents, dry skin care agents, anti-sweat agents, active agents for artificial tanning, glycerol, laponit, caffeine, lipid metabolism regulators, softening, aromatic, refreshing, deodorizing, desensitizing, whitening, scrubbing or nourishing active agents, as well as mixtures of these agents.

Additional active agents may also be selected from agents that improve the barrier function, skin contraction preventing agents, antiglycating agents, agents stimulating the synthesis of dermal and/or epidermal macromolecules and/or preventing their decomposition, agents stimulating fibroblasts or keratinocytes proliferation and/or differentiation of keratinocytes, agents that promote the maturation of the cornified envelope, NO-synthase inhibitors, peripheral benzodiazepine receptor antagonists, agents that increase the activity of the sebaceous glands, agents that stimulate energy metabolism of cells, stretching agents, agents for restructuring fats, agents that promote weight loss, agents that promote capillary blood circulation in the skin, sedatives, agents that regulate the formation of sebum, or anti-seborrheic agents.

In the context of the present invention, the term “microorganism” refers to live microorganisms or their viable forms (e.g., spores), which, when used, can have a positive effect on the skin health, health of the mucous membranes or general health of human or warm-blooded animal, or show antagonistic properties against pathogenic microorganisms, or improve the state of the normal microflora of the body, or representing a bacterial or fungal strain, which is isolated from the skin or mucous membrane of healthy human.

Microorganisms used in the present invention are preferably selected from the group comprising Saccharomyces cerevisiae (including Saccharomyces boulardii), Bacillus subtilis, Bacillus coagulans, Bacillus amyloliquefaciens, Bifidobacterium bifidum, Bifidobacterium longum, Bifidobacterium infantis, Bifidobacterium breve, Bifidobacterium animalis, Bifidobacterium lactis, Bifidobacterium adolescentis, Bifidobacterium pseudocatenulatum, Lactobacillus acidophilus, Lactobacillus alimentarius, Lactobacillus curvatus, Lactobacillus delbruckii subsp. Lactis, Lactobacillus gasseri, Lactobacillus johnsonii, Lactobacillus reuteri, Lactobacillus rhamnosus (GG), Lactobacillus sake, Lactobacillus plantarum, Lactobacillus casei, Lactobacillus paracasei, Lactobacillus kefyr, Lactococcus lactis, Streptococcus thermophilus, Staphylococcus carnosus, Staphylococcus xylosus, Staphylococcus epidermidis, Streptococcus salivarius, Escherichia coli, Propionibacterium (including Propionibacterium freudenreichii) and other members belonging to the types of Actinobacteria, Bacteriodetes, Cyanobacteria, Firmicutes, and Proteobacteria (including their genetically modified strains), as well as their combinations.

The new properties of the microcapsules containing microorganisms provided in the present invention have been identified experimentally.

The microcapsules provided in the present invention can be used as an effective means of delivering live microorganisms to the skin and mucous membranes, when used as an external pharmaceutical or cosmetic product, or as a care product (including oral care products).

The distinctive feature of microcapsules is that they have the form of solid particles containing isolated microorganisms, wherein solid particles disintegrate (soften, melt) when applied to the skin or mucous membranes and release the microorganisms contained in them at the place of their application. Also, microcapsules can have a shell that provides them with additional strength, heat resistance, additional protection from exposure to atmospheric oxygen and aquatic media, but does not prevent the disintegration of microcapsules when applied to the skin or mucous membranes.

The problem is also solved by developing a composition acceptable for use in medicine and cosmetology, which has a new feature of comprising the above-described microcapsules.

It is possible that the composition could have the form of aqueous or anhydrous gel (for example, gel lubricant, scrub gel, deodorant gel or antiperspirant gel, shaving gel or aftershave gel), paste, (for example, toothpaste), foam, ointment, liniment, spray solution, suspension, emulsion, cream mask, cleansing, protective, therapeutic or care cream for face, hands, feet or body (for example, day cream, night cream, makeup remover cream, foundation cream, sunscreen cream), milk or lotion (for example, for shaving or after shaving, for skin care or makeup remover).

It is possible that the composition could have the form of dry powder (for example, tooth powder, powder for inhalation).

Said compositions may also have the form of capsules for application to the skin or mucous membranes, or a pencil or lipstick.

Compositions may have different texture, pH, color, smell and other characteristics and can be used as an external pharmaceutical, cosmetic product or cosmeceutical, care product (including oral care products, skin care product, intimate hygiene product, nasopharynx irrigation product), as well as a toilet product and/or a cosmetic product.

It is advisable that specified composition contains microcapsules in a dosage of from 0.01 to 80 wt. % of the total weight of the composition.

The composition of the powders may include microcapsules in even more quantities—up to 99.99 wt. %.

The microcapsules provided herein and compositions containing said microcapsules can be applied to the skin (on any area of the body skin) or to the mucous membranes (oral, nasal cavity, eyes, genitals) directly from the package or firstly applied to the fingers or palms of the hands, and then applied to the target area by rubbing, which will lead to disintegration of the microcapsules and releasing of microorganisms, they can also be applied using any device—scapula, cotton swab, stick, scrubber, brush, actuator, spraying devices and the like.

The microcapsules provided herein and powder compositions containing thereof can also be applied to the mucous membranes of the nasopharynx and larynx by inhalation or using an inhaler, wherein for this purpose the microcapsules or powder-like composition can be dosed into separate containers (for example, into gelatin capsules), suitable for use in inhaler.

The product provided herein (microcapsules and compositions thereof) may be, in particular, effective for reducing the number of pathogenic microorganisms, improving the microflora balance, homeostasis, barrier function, improving the protective properties and local immunity of the skin and mucous membranes (in particular, for the prevention of dental caries, stomatitis, tonsillitis, pharyngitis, urethrites, etc.), as a products for infectious diseases treating or prevention, cosmetic product or cosmeceutical for prevention and/or treating signs of epidermis aging, for example, wrinkles, little mimic wrinkles, loss of strength, elasticity, density and/or tone of the epidermis, skin discoloration, age-related skin changes, inflammatory manifestations (in particular, comedones or acne), irritation and cracks in the skin and mucous membranes, as well as antiperspirant.

### SUMMARY OF THE DRAWINGS

FIG. 1 shows the view of the microcapsules prepared according to example 1.

FIG. 2 shows the view of the microcapsules prepared according to example 2.

FIG. 3 shows the view of the microcapsules prepared according to example 3.

FIG. 4 shows the view of the microcapsules prepared according to example 4.

FIG. 5 shows the view of the microcapsules prepared according to example 5.

FIG. 6 shows the view of the microcapsules prepared according to example 6.

FIG. 7 shows the view of the microcapsules with the shell prepared according to example 7.

FIG. 8 shows the view of the microcapsules with the shell prepared according to example 8.

FIG. 9 shows the view of the microcapsule with the shell prepared according to example 9.

FIG. 10 shows the view of the microcapsule with the shell prepared according to example 10.

FIG. 11 shows the view of the microcapsules with the shell prepared according to example 11.

The spirit of the invention is illustrated by the examples below.

### EXAMPLE 1. UNCOATED MICROCAPSULES PREPARED BY THE PROCESS OF MECHANICAL GRINDING

50 g of cocoa butter refined (Cargill, USA) with a melting point of 34° C. were melted in a water bath at a temperature of 40° C., 50 g of a mixture of grinded lyophilisate of Saccharomyces cerevisiae (95 wt. %) and sorbitan stearate (5 wt. %) (Angel Yeast Co. Ltd, China) were added and stirred until a homogeneous suspension is formed.

The resulting mixture cooled to room temperature, grinded on a knife mill to a particle size of 100-7000 μm and sieved through a cascade of sieves, taking off fractions with particle size of 100-250 μm, 500-1000 μm, 1000-2000 μm, 2000-3000 μm, 3000-4000 μm, 4000-5000 μm, 5000-6000 μm, 6000-7000 μm.

The obtained fractions were processed on a spheronizer (marumerizer) to provide the microcapsules with a spherical shape.

Uncoated microcapsules containing lyophilisate of live encapsulated microorganisms in the amount of 47.5 wt. % were prepared with a moisture content of 8.7±0.2%.

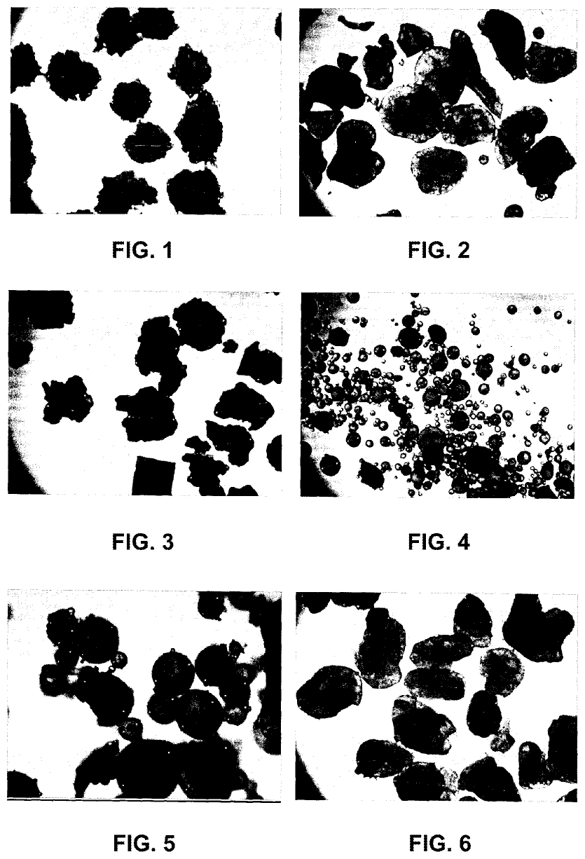

FIG. 1 shows the view of the 500-1000 μm fraction. The view shows that microcapsules have the form of agglomerated particles of the lyophilisate of microorganisms.

Consumer properties of the prepared microcapsules are shown in Table 1.

In alternative embodiments, the homogeneous suspension was cooled below room temperature before grinding.

EXAMPLE 2. UNCOATED MICROCAPSULES PREPARED BY COOLING OF DROPLETS

5 g of white beeswax (Koster Keunen), 2 g of micronized silicon dioxide of the AEROSIL® 200 Pharma trademark (Evonik), 13 g of silicone Cosmetic Grade Fluid of the Dow Corning® 556 trademark and 60 g of silicone Cosmetic Wax of the Dow Corning® 2503 trademark were melted in a water bath at a temperature of 40° C. (softening point of the mixture was 24.7° C.), 20 g of grinded lyophilisate of Bacillus amyloliquefaciens (OOO NPF “Research Center”, Russia) were added and stirred until a homogeneous suspension is formed. The resulting mixture in microdrops (using a micropipette) was added to a liquid cooling agent (liquid nitrogen), the formed microcapsules were separated from the cooling agent, dried and sieved through a sieve, taking off fractions with a particle size of 100-250 μm, 500-1000 μm, 1000-2000 μm, 2000-3000 μm.

Uncoated microcapsules containing lyophilisate of live encapsulated microorganisms in an amount of 20 wt. % were prepared with a moisture content in the microcapsule of 2.4±0.1%.

FIG. 2 shows the view of the 500-1000 μm fraction. The view shows that microcapsules have the form of spherical microparticles containing inclusions of the lyophilisate of microorganisms.

Consumer properties of the prepared microcapsules are shown in Table 1.

Alternative embodiment may use as a liquid cooling agent: cooled water, salt solutions, organic solvent or its mixture with water, CO2.

In alternative embodiments, a pipette, syringe, aspirator, electrospray generator, automatic device for droplets generation, encapsulator or 3D-printer were used for droplets generating.

### EXAMPLE 3. UNCOATED MICROCAPSULES PREPARED BY PROCESS OF AGGLOMERATION IN A FLUIDIZED BED

80 g of lyophilisate of Bifidobacterium bifidum, Bifidobacterium longum (OOO “Bialgam”, Russia) were placed in the working chamber of Mini-Glatt fluidized bed unit (Glatt) and a fluidized bed was formed from the resulting powder. 20 g of a mixture of triglycerides of the Suppocire trademark (Gattefosse) with a melting point of 33.7° C. was melted in a water bath at a temperature of 40° C., 1 mg of β-carotene dye was added, and the fluidized bed was sprayed, for gluing of particles of the probiotic bacteria lyophilisate and forming microcapsules in the form of agglomerates. Then the prepared microcapsules were sieved through a sieve, taking off fractions with a particle size of 100-250 μm, 500-1000 μm, 1000-2000 μm.

Uncoated microcapsules containing lyophilisate of live encapsulated microorganisms in an amount of 80 wt. % were prepared with a moisture content in the microcapsule of 5.7±0.3%.

FIG. 3 shows the view of the 500-1000 μm fraction. The view shows that microcapsules have the form of agglomerated particles of the lyophilisate of microorganisms.

Consumer properties of the prepared microcapsules are shown in Table 1.

### EXAMPLE 4. THE MICROCAPSULES PREPARED BY THE PROCESS OF COOLING AN EMULSION

90 g of a mixture of hydrogenated palm oil (Oleochemicals) with a melting point of 32.9° C. melted in a water bath at a temperature of 45° C., 9.9 g of soybean lecithin and 0.1 g of the ground lyophilisate of Streptococcus thermophilus (FSUE «Experimental Biofabrika», Russia) were added and stirred until a homogeneous suspension is formed. The resulting mixture (homogeneous suspension) was added to a beaker with water (1000 ml) warmed to 40° C., and the aqueous and oil phases intensively stirred until a homogeneous emulsion is formed, lowering the temperature of the resulting emulsion to 4° C. during stirring. Then the stirring was stopped, the resulting solids were separated from the aqueous phase and dried. The resulting powder was sieved through a sieve, taking off fractions with a particle size of 20-125 μm, 125-250 μm, 250-500 μm, 500-1000 μm.

Uncoated microcapsules containing lyophilisate of live encapsulated microorganisms in an amount of 0.1 wt. % were prepared with a moisture content of 3.1±0.1%.

FIG. 4 shows the view of microcapsules prior to fractionation (size of 20-1000 μm). The view shows that microcapsules have the form of microspheres containing inclusions of the lyophilisate of microorganisms.

Consumer properties of the prepared microcapsules are shown in Table 1.

In alternative embodiment, the preparation and subsequent cooling of the emulsion (oil in water) were carried out under controlled conditions using microfluidic grids.

### EXAMPLE 5. MICROCAPSULES PREPARED BY THE PROCESS OF SPRAY COOLING

90 g of a mixture of mono-, di-, and triglycerides of the Witepsol W35 trademark (Oleochemicals) with a melting point of 34.4° C. was melted in a water bath at a temperature of 40° C., 10 g of crushed lyophilisate of Lactobacillus acidophilus, Lactobacillus casei, Lactobacillus plantarum (OOO “Bialgam”, Russia) was added, and stirred until a homogeneous suspension is formed. The resulting mixture was sprayed in a stream of cold air, then the resulting powder was divided in a microparticle classifier, taking off fractions with a particle size of 20-125 μm, 125-250 μm, 250-500 μm, 500-1000 μm.

Uncoated microcapsules containing lyophilisate of live encapsulated microorganisms in the amount of 10 wt. %, were prepared with a moisture content of 1.7±0.1%.

FIG. 5 shows the view of the 500-1000 μm fraction. The view shows that microcapsules have the form of regular-shaped microspheres containing inclusions of the lyophilisate of microorganisms.

Consumer properties of the prepared microcapsules are shown in Table 1.

In alternative embodiments, spraying was performed in a stream of cold nitrogen, gaseous carbon dioxide or inert gas.

In alternative embodiments, spraying was carried out into a liquid cooling agent, which may be chilled water, salt solutions, organic solvent or its mixture with water, liquid nitrogen, or carbon dioxide.

In alternative embodiments, airless spray nozzles, two-, three-, four-, or five-phase nozzles, ultrasonic nozzles, or a rotating disk can be used to spray the suspension.

### EXAMPLE 6. MICROCAPSULES PREPARED BY PROCESS OF HOT EXTRUSION

80 g of a mixture of paraffin cosmetic of the Depilflax trademark with sorbitan stearate (Fine Organics) in a ratio of 19:1 (softening temperature of the mixture is 42° C.) was melted in a water bath at a temperature of 65° C., 20 g of grinded lyophilisate of Escherichia coli (FSUE SPA Microgen, Russia) were added and stirred until a homogeneous suspension is formed. The resulting mixture was cooled to 42-45° C. and pushed through a sieve with a diameter of holes 500 μm. The resulting intermediate was cooled to room temperature and processed on a spheronizer (marumerizer) to provide the particles with spherical shape.

Uncoated microcapsules containing lyophilisate of live encapsulated microorganisms in an amount of 20 wt. % were prepared with a moisture content of 0.9%.

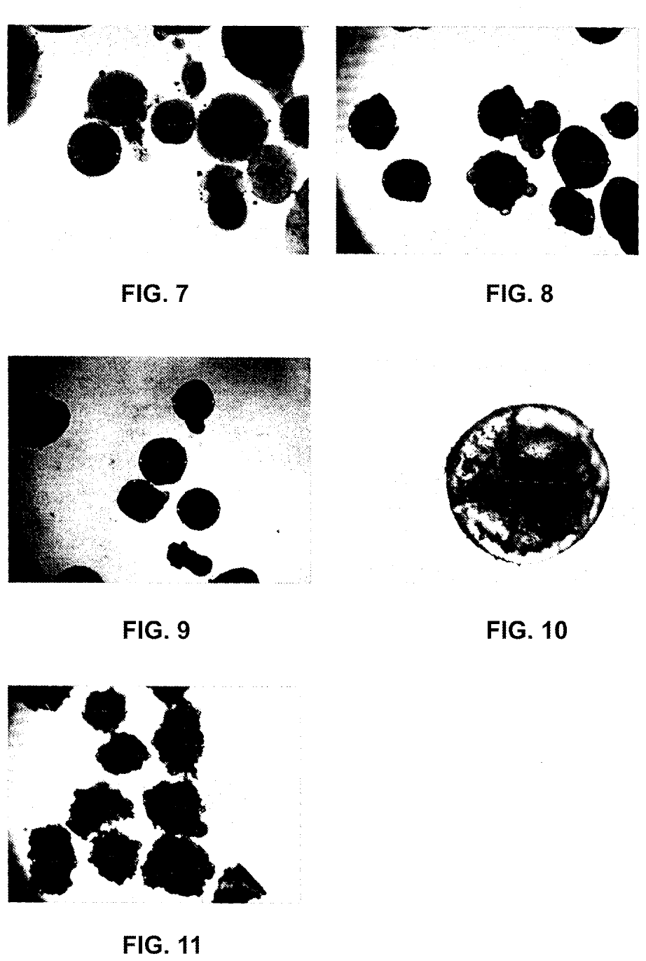

FIG. 6 shows the view of the microcapsules. The view shows that microcapsules have the form of oval-spherical microparticles containing inclusions of the lyophilisate of microorganisms.

Consumer properties of the prepared microcapsules are shown in Table 1.

In alternative embodiments, the process was carried out on an automatic or semi-automatic device—an extruder.

In alternative embodiments, the intermediate was cooled below room temperature.

### EXAMPLE 7. MICROCAPSULES WITH A LIPID SHELL

82 g of microcapsules prepared as described in example 1 were placed in the working chamber of Mini-Glatt unit (Glatt) and a fluidized bed was formed.

18 g of solid fat of the Witepsol E85 trademark (Oleochemicals) with a melting point of 43.1° C. was melted in a water bath at 60° C., 1 mg of (3-carotene dye was added and sprayed into the fluidized bed of the microcapsules for coating.

FIG. 7 shows the view of microcapsules (size of 500-1000 μm). The view shows that microcapsules have the form of microspheres containing a core and a shell.

Consumer properties of the prepared microcapsules are shown in Table 1.

The microcapsules with the shell constituting about 18% of the total weight of the capsule were prepared.

In alternative embodiments, the coating can be applied in a drum coater.

In alternative embodiments, microcapsules prepared according to examples 2-6 can be taken.

### EXAMPLE 8. MICROCAPSULES WITH A WAX SHELL

91 g of microcapsules prepared as described in example 4 were placed in the working chamber of Mini-Glatt unit (Glatt) and a fluidized bed was formed.

9 g of synthetic wax of the Ceralene 692C trademark (Euroceras) with a melting point of 59° C. was melted in a water bath at a temperature of 80° C., 1 mg of β-carotene dye was added and sprayed into the fluidized bed of the microcapsules for coating.

FIG. 8 shows the view of microcapsules (size of 500-1000 μm). The view shows that microcapsules have the form of microspheres coated with the dense shell.

Consumer properties of the prepared microcapsules are shown in Table 1.

The microcapsules with the shell constituting about 9% of the total weight of the capsule were prepared.

In alternative embodiments, microcapsules prepared according to examples 1-3 and 5-7 can be taken.

### EXAMPLE 9. MICROCAPSULES WITH AN ETHYLCELLULOSE SHELL

98 g of microcapsules prepared as described in example 5 (fraction 500-1000 μm), were placed in the working chamber of Mini-Glatt unit (Glatt) and a fluidized bed was formed.

2 g of cellulose ethyl ether of the Ethocel trademark (DOW) were dissolved in 20 ml of 96% ethanol at a temperature of 60° C. with stirring, 1 mg of eosin dye was added, and the resulting solution was sprayed into the fluidized bed of microcapsules for coating.

The microcapsules with the shell constituting about 2% of the total weight of the capsule were obtained.

FIG. 9 shows the view of microcapsules (size of 500-1000 μm). The view shows that microcapsules have the form of microspheres coated with the dense shell.

Consumer properties of the prepared microcapsules are shown in Table 1.

In alternative embodiments, supercritical fluid (carbon dioxide) can be used as a solvent for the shell material.

In alternative embodiments, microcapsules prepared according to examples 1-4 and 6-8 can be taken.

### EXAMPLE 10. MICROCAPSULES WITH HYDROXYPROPYLMETHYLCELLULOSE (HPMC) SHELL

95 g of microcapsules prepared as described in example 6 were placed in the working chamber of Mini-Glatt unit (Glatt) and a fluidized bed was formed.

5 g HPMC of the Vivapharm E6 trademark (JRS Pharm) were dissolved in 50 ml of water, 1 mg of indigo carmine dye was added, and the resulting solution was sprayed into the fluidized bed of microcapsules for coating.

The microcapsules with the shell constituting about 5% of the total weight of the capsule were prepared.

FIG. 10 shows the view of the microcapsule (size of 900 μm). The view shows that microcapsules have the form of microspheres coated with the shell.

Consumer properties of the prepared microcapsules are shown in Table 1.

In alternative embodiments, microcapsules prepared according to examples 1 and 3-9 can be taken.

### EXAMPLE 11. MICROCAPSULES WITH A SHELL OBTAINED BY THE PROCESS OF POWDERING

95 g of microcapsules prepared as described in example 3 (500-1000 μm fraction) and then 5 g of powdering agent (a mixture of micronized cosmetic paraffin of the Depilflax trademark (particle size of 20-40 μm) and micronized magnesium stearate (particle size of 20-30 μm)) were placed in the working chamber of Mini-Glatt unit (Glatt) and a fluidized bed was formed by allowing to stand for 10 minutes at a temperature of 27° C.

FIG. 11 shows the view of microcapsules (size of 500-1000 μm). The view shows that microcapsules have the form of spherical agglomerates.

Consumer properties of the prepared microcapsules are shown in Table 1.

In alternative embodiments, the temperature of the air forming the fluidized bed can be increased to values equal to the melting point of the matrix or shell materials, wherein the temperature increase can be both short-term and permanent.

In alternative embodiments, microcapsules prepared according to examples 1, 2 and 4-10 can be taken.

### EXAMPLE 12. POLYMERIC MICROCAPSULES WITH LACTOBACILLUS

10 g of lyophilisate of Lactobacillus acidophilus, Lactobacillus casei, Lactobacillus plantarum (OOO “Bialgam”, Russia) were dispersed in 20 g of sterile aqueous solution of sodium chloride (0.85 wt. %) and 20 g of sterile aqueous solution of sodium alginate (5 wt. %) were added to this solution. The resulting solution was sprayed into 500 ml of sterile aqueous solution of calcium gluconate (3 wt. %) with the Buchi B-390 encapsulator (nozzle size 500 μm). The prepared microcapsules were separated from the solution, washed with 500 ml of sterile aqueous solution of sodium chloride (0.85 wt. %), frozen at a temperature of −82° C. and lyophilized in TFD-5503 unit (Ilshin).

Uncoated microcapsules containing lyophilisate of live encapsulated microorganisms in the amount of 60 wt. % were prepared with a moisture content of 5.9%.

Consumer properties of the prepared microcapsules are shown in Table 1.

### EXAMPLE 13. POLYMERIC MICROCAPSULES WITH BIFIDOBACTERIUM

The preparation of the microcapsules was carried out as described in example 12, except that lyophilisate of Bifidobacterium bifidum, Bifidobacterium longum (OOO “Bialgam”, Russia) was used.

Uncoated microcapsules containing lyophilisate of live encapsulated microorganisms in an amount of 60 wt. % were prepared with a moisture content of 4.4%.

Views of microcapsules were obtained using an optical microscope Mikmed-5 (JSC “LOMO”). The moisture content was determined on the MA-100 instrument (Sartorius AG).

Evaluation of consumer properties of microcapsules was carried out as follows: 40 g of the microcapsules prepared in examples 1-13 were placed in plastic jars for cosmetics with a screw cap and placed into storage in the climate chamber KFB 115 (Binder) at a temperature of 20±1.0° C. and no light. After 90 days, the jars were opened, and appearance of the microcapsules and the presence or absence of an unpleasant odor were recorded. 200 mg of microcapsules were applied with a spatula as a thin layer on the inner surface of the wrist of the left hand of healthy volunteers (6 persons) with a normal body temperature (36.6-36.7° C.) and kept for 60 seconds (without rubbing on the skin), with observing the changes in the shape and physical state of microcapsules and recording the time of complete deformation from the moment of application to the skin. Then the experiment with rubbing was carried out: 200 mg of microcapsules were applied to the inner surface of the wrist of the left hand of healthy volunteers (6 persons) with a normal body temperature (36.6-36.7° C.) and rubbed on the skin with the palm of the right hand. At the same time, the time of melting of microcapsules and the presence or absence of an abrasive effect (sense of scratching caused by the presence of solid microparticles when rubbing on the skin) were recorded.

Consumer properties of the prepared microcapsules are shown in Table 1.

TABLE 1
Consumer properties of microcapsules prepared according to examples 1-13.

| | | | | | | Disintegrating when | |
|---|---|---|---|---|---|---|---|
| | | | | | | applied to the skin | |
| | | | | | | without | with |
| | | Storage | | Unpleasant | Abrasive | rubbing, | rubbing, |
| No. | Sample | period | Appearance | odor | effect | sec | sec |
| 1 | Microcapsules | 90 days | Brown | no | no | 23 ± 6 | 4 ± 1 |
| | prepared | | agglomerates | | | | |
| | according to | | | | | | |
| | Example 1 | | | | | | |
| | (500-1000 μm | | | | | | |
| | fraction) | | | | | | |
| 2 | according to | 90 days | Cream- | no | no | 13 ± 4 | ≥1 |
| | Example 2 | | coloured | | | | |
| | (500-1000 μm | | microspheres | | | | |
| | fraction) | | | | | | |
| 3 | according to | 90 days | Pink-brown | no | no | 38 ± 10 | 4 ± 1 |
| | Example 3 | | agglomerates | | | | |
| | (500-1000 μm | | | | | | |
| | fraction) | | | | | | |
| 4 | according to | 90 days | Cream- | no | no | 22 ± 2 | 2 ± 1 |
| | Example 4 | | coloured | | | | |
| | (500-1000 μm | | microspheres | | | | |
| | fraction) | | | | | | |
| 5 | according to | 90 days | Yellow | no | no | 19 ± 5 | 3 ± 2 |
| | Example 5 | | microspheres | | | | |
| | (500-1000 μm | | | | | | |
| | fraction) | | | | | | |
| 6 | according to | 90 days | Cream- | no | no | no | 5 ± 2 |
| | Example 6 | | coloured | | | | |
| | (diameter | | microspheres | | | | |
| | 500 μm) | | | | | | |
| 7 | according to | 90 days | Yellow | no | no | no | 7 ± 3 |
| | Example 7 | | microspheres | | | | |
| | (diameter | | | | | | |
| | 500-1000 μm) | | | | | | |
| 8 | according to | 90 days | Yellow | no | no | no | 12 ± 4 |
| | Example 8 | | microspheres | | | | |
| | (diameter | | | | | | |
| | 500-1000 μm) | | | | | | |
| 9 | according to | 90 days | Yellow | no | no | no | 9 ± 3 |
| | Example 9 | | microspheres | | | | |
| | (diameter | | | | | | |
| | 500-1000 μm) | | | | | | |
| 10 | according to | 90 days | Deep blue | no | no | no | 10 ± 4 |
| | Example 10 | | microspheres | | | | |
| | (diameter | | | | | | |
| | 500-1000 μm) | | | | | | |
| 11 | according to | 90 days | Beige- | no | no | 37 ± 12 | 4 ± 2 |
| | Example 11 | | coloured | | | | |
| | (diameter | | microspheres | | | | |
| | 500-1000 μm) | | | | | | |
| 12 | according to | 90 days | Grey-beige | no | yes | no | no |
| | Example 12 | | microspheres | | | | |
| | (diameter | | | | | | |
| | 500 μm) | | | | | | |
| 13 | according to | 90 days | Grey-beige | no | yes | no | no |
| | Example 13 | | microspheres | | | | |
| | (diameter | | | | | | |
| | 500 μm) | | | | | | |

Table 1 shows that the polymer microcapsules prepared according to examples 12 and 13 do not soften and have an abrasive effect when applied to the skin, which makes their use as an external dosage form and cosmetic product unacceptable. The microcapsules prepared according to examples 1-11 have satisfactory consumer properties (no unpleasant odor and no abrasive effect) and are capable to disintegrate when applied to the skin, which indicates their effectiveness as a means of delivering microorganisms for external and cosmetic use. In addition, Table 1 shows that, depending on the nature of the matrix and shell material, microcapsules provided herein (examples 1-11) have different disintegration times when applied to the skin, which makes it possible to use them variably to solve various biopharmaceutical problems related to delivery of live microorganisms to the skin and mucous membranes.

### EXAMPLE 14. COMPARISON OF CONSUMER PROPERTIES AND STABILITY OF THE MICROCAPSULES PROVIDED IN THE PRESENT INVENTION, POLYMERIC MICROCAPSULES AND NON-ENCAPSULATED LYOPHILISATE OF MICROORGANISMS FORMULATED AS A GEL

Consumer properties were evaluated by applying the microcapsules provided according to the present invention prepared as described in example 5 (fraction with size of 500-1000 μm), polymer microcapsules prepared as described in example 12, and non-encapsulated lyophilisate of microorganisms Lactobacillus acidophilus, Lactobacillus casei, Lactobacillus plantarum (manufactured by OOO “Bialgam”, Russia) formulated in an amount of 4 wt. % as a water-containing gel based on polyethylene oxide (this gel can be used as a vehicle for external pharmaceuticals). Then the organoleptic properties and actual number of viable cells of probiotic microorganisms in the gel samples were evaluated during storage at 18° C.

The number of microorganisms in the samples was determined according to GOST R 56139. The comparison results are shown in Table 2.

TABLE 2
Comparison of consumer properties and stability of microcapsules provided in
the present invention, polymeric microcapsules and non-encapsulated lyophilisate of
microorganisms Lactobacillus acidophilus, Lactobacillus casei, Lactobacillus plantarum
formulated as a gel.

| | | | | | | Number of viable |
|---|---|---|---|---|---|---|
| | | Storage | | Unpleasant | Abrasive | microorganisms, |
| No. | Sample | period | Appearance | odor | effect | U/g |
| 1 | Microcapsules | 0 days | Colorless | no | no | 8.8 × 10⁶ |
| | according to the | | transparent gel | | | |
| | present invention | | containing | | | |
| | prepared | | suspended | | | |
| | according to | | spherical | | | |
| | example 5 | | microinclusions | | | |
| | formulated as gel | 5 days | The same | no | no | 8.4 × 10⁶ |
| | | 10 days | The same | no | no | 8.3 × 10⁶ |
| | | 30 days | The same | no | no | 7.5 × 10⁶ |
| | | 60 days | The same | no | no | 7.4 × 10⁶ |
| | | 90 days | The same | no | no | 7.1 × 10⁶ |
| | | 182 days | The same | no | no | 6.8 × 10⁶ |
| | | 364 days | The same | no | no | 6.1 × 10⁶ |
| 2 | Polymer | 0 days | Pinkish | no | yes | 2.4 × 10⁶ |
| | microcapsules | | transparent gel | | | |
| | prepared | | containing | | | |
| | according to | | suspended | | | |
| | example 12 | | spherical | | | |
| | formulated as gel | | microinclusions | | | |
| | | 5 days | Gel turbidity is | weak | — | 8.5 × 10⁵ |
| | | | observed, | | | |
| | | | colour is light | | | |
| | | | brown | | | |
| | | 10 days | Disintegration | Yes | — | 3.3 × 10⁴ |
| | | | of | | | |
| | | | microparticles | | | |
| | | | is observed, gel | | | |
| | | | is turbid and | | | |
| | | | non-transparent, | | | |
| | | | the colour is | | | |
| | | | brown | | | |
| | | 30 days | The same | Yes | — | — |
| 3 | Non-capsulated | 0 days | Non- | no | no | 1.4 × 10⁷ |
| | lyophilis ate of | | transparent | | | |
| | microorganisms | | brown gel with | | | |
| | formulated as gel | | the signs of | | | |
| | layering | | | | | |
| | | 5 days | The same | Yes | no | 8.4 × 10⁴ |
| | | 10 days | The same | Yes | — | 1.3 × 10³ |
| | | 30 days | The same | Yes | — | — |

### EXAMPLE 15. COMPARISON OF CONSUMER PROPERTIES AND STABILITY OF THE MICROCAPSULES PROVIDED IN THE PRESENT INVENTION, POLYMERIC MICROCAPSULES, AND NON-ENCAPSULATED LYOPHILISATE OF MICROORGANISMS FORMULATED AS A CREAM

Consumer properties were evaluated by formulating the microcapsules provided in the present invention prepared as described in example 11, polymer microcapsules, prepared as described in example 13, and non-encapsulated lyophilisate of microorganisms Bifidobacterium bifidum, Bifidobacterium longum (manufactured by OOO “Bialgam”, Russia) as a cosmetic cream (the composition of the cream: purified water, glycerol monostearate, cetyl alcohol, stearyl alcohol, diethylene glycol stearate, PEG-400 stearate, dipropylene glycol, polysorbate 20, PEG-40, hydrogenated castor oil, hydroxypropyl guar, magnesium silicate) in an amount of 4 wt. %. Then the organoleptic properties and actual number of viable cells of probiotic microorganisms in cream samples were evaluated over time during storage at 18° C.

The number of microorganisms in the samples was determined according to GOST R 56139. The comparison results are shown in Table 3.

TABLE 3
Comparison of consumer properties and stability of the microcapsules
provided in the present invention, polymeric microcapsules, and non-encapsulated
lyophilisate of microorganisms Bifidobacterium bifidum, Bifidobacterium longum
formulated as a cream.

| | | | | | Abrasive | |
|---|---|---|---|---|---|---|
| | | | | | effect | |
| | | | | | when | |
| | | | | | applied | Number of viable |
| | | Storage | | Unpleasant | to the | microorganisms, |
| No. | Sample | period | Appearance | odor | skin | U/g |
| 1 | Microcapsules | 0 days | White cream | no | no | 9.5 × 10⁹ |
| | according to the | | containing | | | |
| | present invention | | suspended | | | |
| | prepared | | spherical | | | |
| | according to | | microinclusions | | | |
| | example 11 | 5 days | The same | no | no | 8.6 × 10⁹ |
| | formulated as | 10 days | The same | no | no | 8.2 × 10⁹ |
| | cream | 30 days | The same | no | no | 7.5 × 10⁹ |
| | | 60 days | The same | no | no | 7.4 × 10⁹ |
| | | 90 days | The same | no | no | 7.2 × 10⁹ |
| | | 182 days | The same | no | no | 6.8 × 10⁹ |
| | | 364 days | The same | no | no | 6.3 × 10⁹ |
| 2 | Polymer | 0 days | Light beige | no | yes | 4.3 × 10⁸ |
| | microcapsules | | cream | | | |
| | prepared | | containing | | | |
| | according to | | suspended | | | |
| | example 13 | | spherical | | | |
| | formulated as | | microinclusions | | | |
| | cream | 5 days | Light brown | Yes | — | 4.4 × 10⁷ |
| | | | cream | | | |
| | | 10 days | The same | Yes | — | 8.1 × 10⁴ |
| | | 30 days | The same | Yes | — | — |
| 3 | Non-capsulated | 0 days | Light brown | no | no | 7.0 × 10⁹ |
| | lyophilis ate of | | cream, without | | | |
| | microorganisms | | inclusions | | | |
| | formulated as | 5 days | Browningi s | Weak | — | 3.5 × 10⁵ |
| | cream | | observed | | | |
| | | 10 days | The same | Yes | — | 8.3 × 10³ |
| | | 30 days | The same | Yes | — | — |

Tables 2 and 3 show that non-encapsulated microorganisms formulated as a water-containing gel and cosmetic cream quickly lose their viability (low stability) and cause a change in consumer properties of the dosage form for external use (gel) and cosmetic composition (cream)—discoloration and appearance of an unpleasant odor. Use of polymer microcapsules does not allow to increase the stability of microorganisms and prevent the change in consumer properties of the product. Use of microcapsules provided in the present invention makes it possible to obtain a stable product for external use with satisfactory organoleptic properties, which allows to use microorganisms as an active component in the composition of external pharmaceutical and cosmetic products.

### EXAMPLE 16. MICROCAPSULES PREPARED BY THE PROCESS OF SPRAY COOLING

80 g of saturated fatty acids “Cosmobase A,” with a melting temperature of 29.2° C. (OOO “CosmoLab,” Russia), were melted in a water bath under the temperature of 40° C. 10 g of beeswax (OOO “Pasechnic” Russia) and 10 g of grinded lyophilisate of Staphylococcus Epidermidis (OOO “CosmoLab,” Russia) were added and stirred until a homogeneous suspension is formed. The resulting mixture was sprayed to a liquid cooling agent (liquid nitrogen). The formed microcapsules in liquid nitrogen were placed in a refrigerant at a temperature of 3° C. for 20 hours. The resulting powder was sieved through a cascade of sieves, taking off fractions with particle size of 250-500 μm and 500-1000 μm. Uncoated microcapsules containing lyophilisate of live encapsulated microorganisms in an amount of 10 wt. % were prepared with a moisture content in the microcapsule of 4.9±0.6%.

Consumer properties of microcapsules provided in the Example 16 are set forth in Table 4.

TABLE 4

| | | | | | Abrasive | | |
|---|---|---|---|---|---|---|---|
| | | | | | effect | Disintegrating when | |
| | | | | | when | applied to the skin | |
| | | | | | applied | without | with |
| | | Storage | | Unpleasant | to the | rubbing, | rubbing, |
| No | Sample | period | Appearance | odor | skin | sec | sec |
| 1 | Microcapsules | 90 days | Beige-colored | no | no | 26 ± 4 | 5 ± 2 |
| | prepared | | microspheres | | | | |
| | according to | | | | | | |
| | example 16 | | | | | | |
| | (250-500 μm | | | | | | |
| | fraction) | | | | | | |

Microcapsules containing live microorganisms and their use disclosed in the present invention are intended for use in medicine and cosmetology as a pharmaceutical and cosmetic product for external use for the normalization of microflora and the functional state of the skin and mucous membranes during aging, damage by environmental factors (UV rays, wind, low temperatures, injuries) and pathological processes (microbial infections, inflammatory and allergic diseases, metabolic disorders). At the time of use, when applied to the skin, the microcapsules melt under the action of body temperature, releasing live microorganisms, where the latter have a probiotic effect. In addition, the matrix material, when applied to the skin, has an independent protective, moisturizing and nourishing effect. Use of the microcapsules provided herein makes its possible to isolate microorganisms from water and other active substances contained in the pharmaceutical or cosmetic composition, to prevent the processes of growth of microorganisms inside the package during storage of cosmetic product, thereby eliminating such consumer disadvantages as low stability, short shelf life, unpleasant odor.

1. A microcapsule for a topical treatment for improving skin and mucus membranes condition comprising:
a matrix material in a solid state in which a live probiotic microorganism is encapsulated,
wherein the live microorganism is released from the matrix upon contact with a skin surface and/or mucous membranes of the human or warm-blooded animal body,
wherein the matrix material has a melting or softening at a temperature selected from the range of 20-43° C.-, and
wherein the live probiotic microorganism is present in the range of 0.001 to 80 wt. % from the weight of the matrix material.

2. The microcapsule according to claim 1, wherein the water content in said microcapsule does not exceed 10 wt. %, preferably not more than 5 wt. %, or most preferably does not exceed 1 wt. %.

3. The microcapsule according to claim 1, wherein the matrix material is selected from the group comprising: solid lipids: solid animal and solid vegetable oils and fats, fully hydrogenated or partially hydrogenated vegetable and animal oils and fats, saturated fatty acids, partially hydrogenated or fully hydrogenated fatty acids, fatty acid esters, saturated, partially hydrogenated or fully hydrogenated monoglycerides, diglycerides and triglycerides, phospholipids, lecithins, partially hydrogenated or completely hydrogenated phospholipids and lecithins, lysolecithins and lysophosphatidylcholine; solid waxes: animal waxes, plant waxes, mineral waxes, synthetic waxes, wax esters, saturated and unsaturated fatty alcohols, fatty alcohol ethers/esters; solid saturated and unsaturated hydrocarbons (paraffins); solid silicones and silicone ethers/esters; polyol ethers/esters: glycerol ethers/esters, sorbitan, sorbitan stearate, glyceryl ricinoleate; polyglycerols and their ethers/esters, hydrophobic gelling agents: silicon dioxide, polyethylenes, and their mixtures.

4. The microcapsule according to claim 1, wherein the melting or softening point of the matrix is selected from the range of 26.5-35.0° C.

5. The microcapsule according to claim 1, wherein the microcapsule has a size of 100-7000 μm and is prepared by mechanical grinding of a cooled suspension of the probiotic microorganism in the matrix material.

6. The microcapsule according to claim 1, wherein the microcapsule have has a size of 50-3000 μm and is prepared by cooling of droplets of a suspension of the probiotic microorganism in the matrix material.

7. The microcapsule according to claim 1, wherein the microcapsule has a size of 100-2000 μm and are is prepared by spraying the molten matrix material into the fluidized bed of the lyophilisate of the probiotic microorganism.

8. The microcapsule according to claim 1, wherein the microcapsule has a size of 20-1000 μm and are is prepared by cooling of emulsified suspension of the probiotic microorganism in the matrix material.

9. The microcapsule according to claim 1, wherein the microcapsule have has a size of 20-1000 μm and are prepared by cooling of the sprayed suspension of the lyophilisate of the probiotic microorganism in the matrix material.

10. The Microcapsule according to claim 1, wherein the microcapsule has a size of 250-5000 μm and are is prepared by hot extrusion of the suspension of the probiotic microorganism in the matrix material.

11. The microcapsule according to claim 1, wherein the microcapsule additionally contains at least one shell and/or coating that melt or disintegrate when the microcapsule is applied to the skin or mucous membranes.

12. The microcapsule according to claim 11, wherein the shell is obtained by spraying a molten shell material on the microcapsule.

13. The microcapsule according to claim 12, wherein the melting point of the shell material is selected from a range of 28-72° C., preferably 35.5-54° C.

14. The microcapsule according to claim 13, wherein the shell material has the same melting point as the matrix material.

15. The microcapsule according to claim 14, wherein the amount of the shell material does not exceed 50% of the total weight of the microcapsule.

16. The microcapsule according to claim 13, wherein the shell material has a melting point exceeding the melting point of the matrix material.

17. The microcapsule according to claim 16, wherein the amount of the shell material does not exceed 20% of the total weight of the microcapsule.

18. The microcapsule according to claim 11, wherein the shell is obtained by spraying a solution or suspension of the shell material on the microcapsule.

19. The microcapsule according to claim 18, wherein the shell material is selected from the group comprising the same substances as the matrix material.

20. The microcapsule according to claim 18, wherein the shell material is selected from the group comprising cellulose ethers:
hydroxymethylpropylcellulose (HPMC) and its derivatives, hydroxypropylcellulose, methylcellulose, ethylcellulose, carboxymethylcellulose (CMC), cellulose acetate phthalate, methacrylic acid and its derivatives (Eudragit), polyvinylpyrrolidone and its derivatives, polysaccharides and their derivatives: sodium alginate, gum arabic, gellan gum, starch, modified starch, guar gum, pectin, amidated pectin, carrageenan, chitosan, mesquite gum, agar gum, psyllium gum, tamarind gum, xanthan, locust bean gum; protein: wheat protein, soy protein, sodium caseinate, gelatin, zein, shellac, hyaluronic acid and its derivatives, any synthetic and natural water-soluble polymers, and mixtures thereof.

21. The microcapsule according to claim 20, wherein the amount of the shell material does not exceed 20% of the total weight of the microcapsule.

22. The microcapsule of claim 11, wherein the coating is obtained by powdering the microcapsule with a micronized coating material.

23. The microcapsule according to claim 22, wherein the micronized coating material is selected from the group of substances comprising the same substances as the matrix material or the shell material; or from the groups of substances comprising inorganic salts, metal oxides, talc, salts and esters of saturated and unsaturated fatty acids.

24. The microcapsule according to claim 1, wherein the microcapsule additionally contain at least one coloring agent and/or at least one substance suitable for use in pharmaceutical, food and cosmetic products.

25. The microcapsule according to claim 1, wherein the probiotic microorganism is selected from the group comprising Saccharomyces cerevisiae (including Saccharomyces boulardii), Bacillus subtilis, Bacillus coagulans, Bacillus amyloliquefaciens, Bifidobacterium bifidum, Bifidobacterium longum, Bifidobacterium infantis, Bifidobacterium breve, Bifidobacterium animalis, Bifidobacterium lactis, Bifidobacterium adolescentis, Bifidobacterium pseudocatenulatum, Lactobacillus acidophilus, Lactobacillus alimentarius, Lactobacillus curvatus, Lactobacillus delbruckii subsp. Lactis, Lactobacillus gasseri, Lactobacillus johnsonii, Lactobacillus reuteri, Lactobacillus rhamnosus (GG), Lactobacillus sake, Lactobacillus plantarum, Lactobacillus casei, Lactobacillus paracasei, Lactobacillus kefyr, Lactococcus lactis, Streptococcus thermophilus, Staphylococcus carnosus, Staphylococcus xylosus, Staphylococcus epidermidis, Streptococcus salivarius, Escherichia coli, Propionibacterium (including Propionibacterium freudenreichii), or members belonging to the types of Actinobacteria, Bacteriodetes, Cyanobacteria, Firmicutes, and Proteobacteria (including their genetically modified strains), as well as their combinations.

26. Use of the microcapsule according to claim 1 as an external pharmaceutical, cosmetic, cosmeceutical product for treatment of skin or mucous membrane of the subject in need thereof.

27. A composition containing the microcapsule according to claim 1, suitable for use as external pharmaceutical, cosmetic, cosmeceutical product for treatment of skin or mucous membrane of the subject in need thereof.

28. The composition according to claim 27, wherein composition contains the microcapsule in a dosage of 0.01 to 80 wt. % of the total weight of the composition.

29. The composition according to claim 27, wherein the composition has the form of a cream, lotion, gel, paste, solution, suspension, ointment, emulsion, or powder.
